(19) United States
(12) Patent Application Publication
(10) Pub. No.: US 2012/0020901 A1
(43) Pub. Date: Jan. 26, 2012

(54) ORAL CARE COMPOSITIONS

(75) Inventor: Ben Gu, East Brunswick, NJ (US)

(73) Assignee: Colgate-Palmolive Company, New York, NY (US)

(21) Appl. No.: 13/262,353

(22) PCT Filed: Apr. 2, 2009
(86) PCT No.: PCT/US2009/039311

§ 371 Date: Sep. 30, 2011

(51) Int. Cl. A61K 8/55; A61Q 11/00

(52) U.S. Cl. 424/57

(57) ABSTRACT

The invention relates to desensitizing, anti-tartar oral care compositions comprising an orally acceptable vehicle and an effective desensitizing and anti-tartar amount of tetrapotassium pyrophosphate.

### BACKGROUND OF THE INVENTION

Dentinal hypersensitivity represents a lowering tooth pain threshold due to pulpal inflammation. Dentinal hypersensitivity can cause acute tooth pain in response to thermal, osmotic and electrical stimulations, and may be experienced by up to 30% of the population.

There are various regimens to treat dentinal hypersensitivity. An over-the-counter (OTC) desensitizing dentifrice is the most popular choice for the treatment of sensitive teeth. Various chemical constituents have been investigated for the treatment of hypersensitive teeth and have showed clinical efficacy to alleviate dentinal hypersensitivity, and have been incorporated into OTC desensitizing dentifrices. Particularly widely used desensitizing agents are strontium chloride and potassium nitrate. In an earlier clinical study over a period of eight weeks, a dentifrice incorporating 5 wt % potassium nitrate was found to be significantly superior in reducing teeth sensitivity than a dentifrice incorporating 10 wt % strontium chloride.

Accordingly, potassium nitrate is formulated into many commercially available desensitizing dentifrices.

Dental calculus, also called tartar or calcified plaque, is a primary factor to cause periodontal disease, and is very prevalent, typically being present in up to 90% of the population. Dental calculus can be removed by a dentist and can also be prevented by using a dentifrice containing an anti-tartar agent, acting as a tartar inhibitor, to minimize the deposition of calculus on the tooth surface.

In many studies, tetrasodium pyrophosphate has been shown to constitute an effective calculus inhibitor, and is widely used in commercial tartar control dentifrices.

U.S. Pat. No. 4,931,273 discloses an anticalculus dentifrice that contains as anticalculus agent comprising 4.3 to 7 wt % of alkali metal pyrophosphates comprising at least 4.3 wt % tetrapotassium pyrophosphate alone or admixed with up to 2.7 wt % tetrasodium pyrophosphate.

U.S. Pat. No. 5,505,933 discloses a desensitizing anti-tartar dentifrice that contains an effective anti-tartar proportion of an anti-tartar agent, which may be tetrapotassium pyrophosphate, and a desensitizing potassium salt, such as potassium nitrate, potassium citrate, potassium oxalate or a mixture of two or more thereof. In one Example, a dentifrice contains, inter alia, 2.5 wt % tetrapotassium pyrophosphate and 5 wt % potassium nitrate.

U.S. Pat. No. 5,374,417 and U.S. Pat. No. 5,486,350 each disclose a desensitizing antitartar dentifrice that contains tetrapotassium pyrophosphate as an anti-tartar agent and a potassium salt, such as potassium nitrate, potassium citrate, potassium oxalate or a mixture of two or more thereof as a desensitizing agent. In one Example, a dentifrice contains, inter alia, 2.5 wt % tetrapotassium pyrophosphate and 5 wt % potassium nitrate.

Despite the known use of tetrapotassium pyrophosphate as an anti-tartar agent, a need exists to provide desensitizing oral care compositions, in particular dentifrices, exhibiting good desensitizing and tartar control benefits which can be produced cost effectively.

### BRIEF SUMMARY OF THE INVENTION

The invention relates to oral care compositions, and in particular such compositions providing the combination of desensitizing and tartar control benefits.

The invention includes a desensitizing, anti-tartar oral care composition comprising an orally acceptable vehicle and an effective desensitizing and anti-tartar amount of tetrapotassium pyrophosphate.

The tetrapotassium pyrophosphate may be present in an amount of from from about 1 to about 8 wt %, preferably about 3 to about 6 wt %, more preferably about 4 to about 5 wt. % based on the weight of the composition. Typically, the tetrapotassium pyrophosphate is present in an amount of about 4 wt % based on the weight of the composition.

The tetrapotassium pyrophosphate may comprise the sole potassium salt in the composition.

The composition may further comprise an anti-calculus amount of sodium tripolyphosphate. The sodium tripolyphosphate may be present in an amount of from about 1 to about 10 wt % based on the weight of the composition, more preferably about 1 to about 8 wt % based on the weight of the composition. Typically, the tetrasodium tripolyphosphate is present in an amount of about 7 wt % based on the weight of the composition.

Embodiments of the present invention also provide a method of removing tartar from a dental surface, the method comprising contacting a dental surface of the oral cavity with the oral care composition of the present invention.

Embodiments of the present invention also provide a method of removing tartar from a dental surface, the method comprising contacting a dental surface of the oral cavity with the oral care composition of the present invention.

Embodiments of the present invention also provide a method of treating sensitive teeth and removing tartar from a tooth surface, the method comprising contacting a sensitive tooth surface with the oral care composition of the present invention.

The invention also includes a desensitizing, anti-tartar oral care composition comprising, as a dual-function active agent providing desensitizing and anti-tartar control benefits, an effective amount of tetrapotassium pyrophosphate in an orally acceptable vehicle.

The invention further includes the use, in a desensitizing, anti-tartar oral care composition, of tetrapotassium pyrophosphate as a dual-function active agent providing desensitizing and anti-tartar control benefits.

### DETAILED DESCRIPTION OF THE INVENTION

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

The invention broadly encompasses a desensitizing, anti-tartar oral care composition comprising an orally acceptable vehicle and an effective desensitizing and anti-tartar amount of tetrapotassium pyrophosphate.

The present invention is predicated on the finding by the present inventor that tetrapotassium pyrophosphate can provide a dual function within an oral care composition, in particular a dentifrice, namely a first function of providing a desensitizing benefit and a second function of providing a tartar control benefit. These two functions can be achieved by the single active agent of tetrapotassium pyrophosphate. There is no need to provide an additional potassium-based desensitizing agent.

This technical solution of a dual-function additive also provides significant cost savings as compared to known dentifrices providing a desensitizing benefit. This is because in order to provide a given concentration of potassium ion within the dentifrice to provide a given desensitizing benefit, typically about 1.95% potassium ion based on the weight of the dentifrice composition, the cost of using tetrapotassium pyrophosphate as the sole potassium source is significantly lower than if the potassium source is potassium nitrate (approximately four times more expensive), potassium citrate (approximately two times more expensive) or potassium chloride (approximately two and a half times more expensive).

The tetrapotassium pyrophosphate may be present in an amount of from about 3 to about 6 wt %, more preferably about 4 to about 5 wt % based on the weight of the composition. Typically, the tetrapotassium pyrophosphate is present in an amount of about 4 wt % based on the weight of the composition.

The tetrapotassium pyrophosphate may comprise the sole potassium salt in the composition.

The composition may further comprise an anti-calculus amount of tetrasodium tripolyphosphate. The tetrasodium tripolyphosphate may optionally be present at a relatively low level to provide additional calculus inhibiting benefits in addition to the tetrapotassium pyrophosphate. The tetrasodium tripolyphosphate may be present in an amount of from about 1 to about 5 wt % based on the weight of the composition. Typically, the tetrasodium tripolyphosphate is present in an amount of about 2 wt % based on the weight of the composition.

The oral care composition of the present invention may be used in a method of removing tartar from a dental surface, the method comprising contacting a dental surface of the oral cavity with the oral care composition of the present invention.

The oral care composition of the present invention may be used in a method of treating sensitive teeth, the method comprising contacting a sensitive tooth surface with the oral care composition of the present invention.

The oral care composition of the present invention may be used in a method of treating sensitive teeth and removing tartar from a tooth surface, the method comprising contacting a sensitive tooth surface with the oral care composition of the present invention.

The invention also includes a desensitizing, anti-tartar oral care composition comprising, as a dual-function active agent providing desensitizing and anti-tartar control benefits, an effective amount of tetrapotassium pyrophosphate in an orally acceptable vehicle.

The invention further includes the use, in a desensitizing, anti-tartar oral care composition, of tetrapotassium pyrophosphate as a dual-function active agent providing desensitizing and anti-tartar control benefits.

The tetrapotassium pyrophosphate in the oral care compositions of the invention may act as a chelating agent able to complex calcium found in the cell walls of bacteria. Binding of this calcium weakens the bacterial cell wall and augments bacterial lysis. The tetrapotassium pyrophosphate in both hydrated and unhydrated form. An effective amount of pyrophosphate salt useful as such a chelating agent in the present composition is generally enough to provide at least 1.0% pyrophosphate ions, from about 1.5% to about 6%, from about 3.5% to about 6% of such ions.

The oral care compositions of preferred embodiments into which the tetrapotassium pyrophosphate is incorporated comprise a carrier which can include various ingredients such as at least one abrasive, at least one fluoride source, at least one surfactant, at least one vitamin, at least one polymer, at least one flavoring agent; at least one enzyme, at least one humectant, and/or at least one preservative and combinations thereof. Food-grade tetrapotassium pyrophosphate for use in the compositions of the invention is widely available in commerce.

In certain embodiments, an abrasive is present in the oral care composition in an amount of about 1 to 50 wt. %. In certain embodiments, the fluoride source is present in an amount of about 0.01 to 5 wt. %. In certain embodiments, the flavoring agent in an amount of about 0.01 to 5 wt. %. In certain embodiments, the tape or strip further includes at least one humectant in an amount of about 0.01 to 40 wt. %.

The oral care compositions may further include one or more fluoride ion sources. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., incorporated herein by reference.

Representative fluoride ion sources include, but are not limited to, stannous fluoride, sodium fluoride, potassium fluoride, sodium monofluorophosphate, sodium fluorosilicate, sodium monfluorophosphate (MFP), ammonium fluorosilicate, as well as tin fluorides, such as stannous fluoride and stannous chloride, and combinations thereof. Certain particular embodiments include stannous fluoride or sodium fluoride as well as mixtures thereof.

In certain embodiments, the oral care composition of the invention may also contain a source of fluoride ions or fluorine-providing ingredient in amounts sufficient to supply about 25 ppm to 5,000 ppm of fluoride ions.

Fluoride ion sources may be added to the compositions of the invention at a level of from about 0.01% to 3.0% in one embodiment or from about 0.03% to 1.0%, by weight of the composition in another embodiment.

Another agent optionally included in the composition may be a surfactant or a mixture of compatible surfactants. Suitable surfactants are those which are reasonably stable throughout a wide pH range, for example, anionic, cationic, nonionic or zwitterionic surfactants.

Suitable surfactants are described more fully, for example, in U.S. Pat. No. 3,959,458, to Agricola et al.; U.S. Pat. No. 3,937,807, to Haefele; and U.S. Pat. No. 4,051,234, to Gieske et al., which are incorporated herein by reference.

In certain embodiments, the anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate, sodium lauroyl sarcosinate and sodium coconut monoglyceride sulfonates are examples of anionic surfactants of this type. Mixtures of anionic surfactants may also be utilized.

In another embodiment, cationic surfactants useful can be broadly defined as derivatives of aliphatic quaternary ammonium compounds having one long alkyl chain containing from about 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof.

Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants that can be used in the compositions can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

In certain embodiments, zwitterionic synthetic surfactants may be used and can be broadly described as derivatives of aliphatic quaternary ammonium, phosphomium, and sulfonium compounds, in which the aliphatic radicals can be straight chain or branched, one of the aliphatic substituents can contain from about 8 to 18 carbon atoms and contain an anionic water-solubilizing group, e.g., carboxy, sulfonate, sulfate, phosphate or phosphonate. Illustrative examples of the surfactants suited for inclusion into the composition include, but are not limited to, sodium alkyl sulfate, sodium lauroyl sarcosinate, cocoamidopropyl betaine and polysorbate 20, and combinations thereof.

The surfactant or mixtures of compatible surfactants can be present in the compositions of the present invention from about 0.1% to about 5.0%, in another embodiment from about 0.3% to about 3.0% and in another embodiment from about 0.5% to about 2.0% by weight of the total composition. The dosage of surfactant in the individual strip or tape (i.e., a single dose) is about 0.001 to 0.05% by weight, 0.003 to 0.03% by weight, and in another embodiment about 0.005 to 0.02% by weight.

Flavoring agents which are used in the practice of the present invention include, but are not limited to, essential oils as well as various flavoring aldehydes, esters, alcohols, and similar materials. Examples of the essential oils include oils of spearmint, peppermint, wintergreen, sassafras, dove, sage, eucalyptus, marjoram, cinnamon, lemon, lime, grapefruit, and orange. Also useful are such chemicals as menthol, carvone, and anethole. Certain embodiments employ the oils of peppermint and spearmint.

The compositions also optionally include one or more polymers. Such materials are well known in the art, being employed in the form of their free acids or partially or fully neutralized water soluble alkali metal (e.g. potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight from 1,000-2,000,000, described in U.S. Pat. No. 4,842,847, Jun. 27, 1989 to Zahid, incorporated herein by reference.

Another useful class of polymeric agents includes polyamino acids, particularly those containing proportions of anionic surface-active amino acids such as aspattic acid, glutamic acid and phosphoserine, as disclosed in U.S. Pat. No. 4,866,161 Sikes et al., incorporated herein by reference.

The oral care compositions of the invention may also optionally include one or more enzymes. Useful enzymes include any of the available proteases, glucanohydrolases, endoglycosidases, amylases, mutanases, lipases and mucinases or compatible mixtures thereof. In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. No. 5,000,939 to Dring et al., U.S. Pat. No. 4,992,420; U.S. Pat. No. 4,355,022; U.S. Pat. No. 4,154,815; U.S. Pat. No. 4,058,595; U.S. Pat. No. 3,991,177; and U.S. Pat. No. 3,696,191 all incorporated herein by reference. An enzyme of a mixture of several compatible enzymes in the current invention constitutes from about 0.002% to about 2.0% in one embodiment or from about 0.05% to about 1.5% in another embodiment or in vet another embodiment from. about 0.1% to about 0.5%.

Water may also be present in the oral compositions of the invention. Water, employed in the preparation of commercial oral compositions should be deionized and free of organic impurities. Water commonly makes up the balance of the compositions and includes from about 10% to 50%, about 20% to 40% or about 10% to 15% by weight of the oral compositions. This amount of water includes the free water which is added plus that amount which is introduced with other materials such as with sorbitol or the tetrapotassium pyrophosphate component of the invention.

In preparing oral care compositions, it is sometimes necessary to add some thickening material to provide a desirable consistency. In certain embodiments, the thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. Thickening agents in an amount from 0.5% to 5.0% by weight of the total composition can be used.

Within certain embodiments of the oral compositions, it is also desirable to incorporate a humectant to prevent the composition from hardening upon exposure to air. Certain humectants can also impart desirable sweetness or flavor to dentifrice compositions. The humectant, on a pure humectant basis, generally includes from about 15% to 70% in one embodiment or from about 30% to 65% in another embodiment by weight of the dentifrice composition.

Suitable humectants include edible polyhydric alcohols such as glycerine, sorbitol, xylitol, propylene glycol as well as other polyols and mixtures of these humectants. Mixtures of glycerine and sorbitol may be used in certain embodiments as the humectant component of the toothpaste compositions herein.

In addition to the above described components, the embodiments of this invention can contain a variety of optional dentifrice ingredients some of which are described below. Optional ingredients include, for example, but are not limited to, adhesives, sudsing agents, flavoring agents, sweetening agents, additional antiplaque agents, abrasives, and coloring agents. These and other optional components are further described in U.S. Pat. No. 5,004,597, to Majeti; U.S. Pat. No. 3,959,458 to Agricola et al. and U.S. Pat. No. 3,937,807, to Haefele, all being incorporated herein by reference.

A number of references have been cited, the entire disclosures of which are incorporated herein by reference.

We claim:

1. A desensitizing, anti-tartar oral care composition comprising an orally acceptable vehicle and an effective desensitizing and anti-tartar amount of tetrapotassium pyrophosphate.

2. The composition of claim 1, wherein the tetrapotassium pyrophosphate is present in an amount of from about 1 to about 8 wt % based on the weight of the composition.

3. The composition of claim 2, wherein the tetrapotassium pyrophosphate is present in an amount of from about 3 to about 6 wt % based on the weight of the composition.

4. The composition of claim 3, wherein the tetrapotassium pyrophosphate is present in an amount of about 4 to about 5 wt % based on the weight of the composition.

5. The composition of claim 4, wherein the tetrapotassium pyrophosphate is present in an amount of about 4 wt % based on the weight of the composition.

6. The composition of claim 1, wherein the tetrapotassium pyrophosphate comprises the sole potassium salt in the composition.

7. The composition of claim 1, further comprising an anti-calculus amount of sodium tripolyphosphate.

8. The composition of claim 7, wherein the sodium tripolyphosphate is present in an amount of from about 1 to about 10 wt % based on the weight of the composition.

9. The composition of claim 8, wherein the sodium tripolyphosphate is present in an amount of about 7 wt % based on the weight of the composition.

10. A method of removing tartar from a tooth surface, the method comprising contacting a tooth surface of the oral cavity with the oral care composition of claim 1.

11. A method of treating sensitive teeth, the method comprising contacting a sensitive tooth surface with the oral care composition of claim 1.

12. A method of treating sensitive teeth and removing tartar from a tooth surface, the method comprising contacting a sensitive tooth surface with the oral care composition of claim 1.

13. A desensitizing, anti-tartar oral care composition comprising, as a dual-function active agent providing desensitizing and anti-tartar control benefits, an effective amount of tetrapotassium pyrophosphate in an orally acceptable vehicle.

14. The composition of claim 13, wherein the tetrapotassium pyrophosphate is present in an amount of about 4 wt % based on the weight of the composition.

15. The composition of claim 14, wherein the tetrapotassium pyrophosphate comprises the sole potassium salt in the composition.

16. Use, in a desensitizing, anti-tartar oral care composition, of tetrapotassium pyrophosphate as a dual-function active agent providing desensitizing and anti-tartar control benefits.

17. The use of claim 16, wherein the tetrapotassium pyrophosphate is present in an amount of about 4 wt % based on the weight of the composition.

18. The use of claim 17, wherein the tetrapotassium pyrophosphate comprises the sole potassium salt in the composition.
